(19) United States
(12) Patent Application Publication
(10) Pub. No.: US 2007/0184460 A1
(43) Pub. Date: Aug. 9, 2007

(54) Diagnostic assay for Orientia tsutsugamushi by detection of responsive gene expression

(76) Inventors: Wei-Mei Ching, Bethesda, MD (US); Xuan Li, Silver Spring, MD (US)

Correspondence Address: NAVAL MEDICAL RESEARCH CENTER;ATTN: (CODE 00L), 503 ROBERT GRANT AVENUE, SILVER SPRING, MD, 20910-7500, US

(21) Appl. No.: 11/357,462

(22) Filed: Feb. 9, 2006

(51) Int. Cl. C12Q 1/68; C12P 19/34

(52) U.S. Cl. 435/6; 435/91.2

(57) ABSTRACT

The inventive subject matter relates to a method for the diagnosis of Orientia tsutsugamushi infection by measuring the increased or decreased expression of specific human genes following infection by microarray or polymerase chain reaction analysis. The method employs the creation of gene modulation profiles in patients suspected to be infected with O. tsutsugamushi and comparing the profiles with a pre-determined profile of genes known to modulate in response to O. tsutsugamushi exposure and infection. The method permits the early detection of O. tsutsugamushi infection and diagnosis of scrub typhus earlier than currently available methods. The method also permits mid-course monitoring of disease progression with greater detail than currently available methods.

### BACKGROUND OF INVENTION

1. Field of Invention

The inventive subject matter relates to a method of diagnosing Rickettsial diseases by analysis of modulation of host gene expression. The method contemplates the use of microarray technology for the detection and analysis of gene up or down regulation in response to bacterial infection.

2. Description of Related Art

The disease scrub typhus, caused by the Gram negative bacteria Orientia (formerly Rickettsia) tsutsugamushi is one of the most common rickettsial diseases and can cause up to 35% mortality if left untreated (1, 2). The bacterial pathogen accounts for up to 23% of all febrile episodes in endemic areas of the Asia-Pacific region. Geographic distribution of the disease occurs principally within an area of about 13 million square kilometers and includes Pakistan, India and Nepal in the west to Japan in the east and from southeastern Siberia, China and Korea in the north to Indonesia, Philippines, northern Australia and the intervening Pacific islands in the south. During World War II, more than 5,000 cases of scrub typhus were reported among U.S. troops and 30,000 cases for Japanese troops. Scrub typhus ranked only behind malaria as the most important arthropod borne infectious disease. More recently, scrub typhus was the second leading cause of fevers of unknown origin among U.S. personnel during the Vietnam conflict.

Because of the relatively high mortality rate in untreated patients, the rising prevalence of drug resistant strains, and the lack of vaccines against the organism, early detection of exposure and infection is becoming increasingly important. For this reason, simple and accurate methods are important for early detection and effective treatment of the disease. However, despite the global public health importance of scrub typhus, currently available diagnostic methods are inadequate. Diagnosis of scrub typhus is generally based on the clinical presentation and history of the patient. Because of similarities in symptomatology, however, differentiation of scrub typhus from other febrile diseases, such as leptospirosis, murine typhus, malaria, dengue fever and viral hemorrhagic fevers, is often difficult especially early after infection.

In order to overcome the short-comings in scrub typhus diagnosis, significant research effort has been devoted to developing accurate laboratory diagnostic methods for scrub typhus. The currently available assays are typically seriologically-based and include indirect-fluorescence assay (IFA), indirect immunoperoxidase assay (IIP), enzyme-linked immunosorbent assay (ELISA) and dot blot assays. These assays, however all suffer from the requirement of requiring the availability of antigen which typically entails growing rickettsiae grown in host cells or preparing extracts of purified bacteria as well as the availability of antibody in patient sera (3-10). Additionally, the assay methods are time consuming to perform and offer limited insight into serotypes not represented by the panel of available antigen.

A problematic hurdle in the design of sensitive and accurate diagnostic assays is ensuring the assay's effectiveness early after infection. In currently available and employed antibody-based assays, sensitivity requires a suitable number of bacteria in tissue samples. Typically, adequate levels of bacterial load to meet the required threshold are not found, especially early after an infection. Likewise, detection of seroconversion is also not an effective diagnostic method early after exposure and infection since no detectable, specific antibody would be present.

Other confounding issues in designing suitable assays include the fact that Orientia strains exhibit significant antigenic differences thereby complicating assay antigen selection for use in available scrub typhus serodiagnostic procedures. For example, the major outer membrane protein (vOmp) of O. tsutsugamushi is an important serodiagnostic antigen but varies from 53-63 kDa even among isolates from the same country (11). Furthermore, both unique and cross-reactive domains exist in different homologs that potentially necessitating the use of multiple strains in scrub typhus diagnostic test design. Additionally, the list of scrub typhus serotypes is incomplete.

Polymerase chain reaction (PCR) amplification of O. tsutsugamushi genes has been demonstrated to be a reliable diagnostic method for scrub typhus (12, 13). PCR permits the rapid identification of distinct genetypes that are associated with Orienta serotypes (12, 14-18). However, despite the advantages of PCR, significant disadvantages include the requirement for sophisticated instrumentation and labile reagents to conduct the assays that are often not available in rural medical facilities. Additionally, PCR procedures are highly susceptible to false positive results due to inadvertent carry-over of nucleic acid material. This is particularly prevalent in field settings or in facilities that are not fully equipped to conduct PCR laboratory procedures.

A solution to the paucity of early diagnostic methods is to monitor the expression of host response genes in response to infection. Early after exposure to an infectious organism, host responsiveness to infection is manifested by modulation of specific gene expression. Some genes are differentially expressed very early after infection thus permitting the construction of unique gene expression profiles that are exhibited early after infection of human cells, such as peripheral blood mononuclear cells (PBMC). The patterns or profiles of gene expression would thus enable the differentiation of exposure by pathogens and toxins, including Bacillus anthracis, Yersinia pestis, Brucella melitensis, botulinum toxin, staphylococcal exotoxins A and B (SEB, SEA), lipopolysaccharide (LPS), cholera toxin, Venezuelan equine encephalitis virus (19). Furthermore, it has been previously shown that specific human genes modulate up or down in response to bacterial infection (20).

Semi-quantitative reverse transcriptase polymerase chain reaction (RT-PCR) is capable of sensitively measuring changes in gene expression from collected host cell RNA. By designing primer sets specific to a limited number of genes, known to have altered expression following infection, molecular-based assays can be devised to diagnosis and monitor infection early after infection by direct assessment of gene modulation.

Although measurement of changes in gene expression by RT-PCR is a valuable diagnostic strategy, the method suffers from the disadvantages associated with PCR in that it is often not suitable for high-throughput screening of large numbers of genes. A more convenient method of measuring gene expression changes is by hybridizing amplified RNA onto cDNA microarrays containing large numbers of double-stranded sequences of important host genes. A number of computer programs are available to accurately analyze and transform the ensuing gene expression data into useful and reproducible gene expression profiles.

Microarrays are well suited for high-throughput detection of thousands of differentially expressed genes in a single experiment (21). The method allows for the characterization of the cascade of cellular signaling and concomitant interrelated host gene expression profiles following infection by specific pathogens or toxins (22, 23). Therefore, data from cDNA microarrays provides the ability to quickly and accurately assess and monitor the changes in gene expression profiles specific to infection by specific pathogenic organisms. Microarrays can also be used to evaluate genomic differences between virulent and nonvirulent strains of a species (24).

Therefore, in order to improve early diagnosis of scrub typhus, an aspect of this invention is the diagnosis of O. tsutsugamushi early after exposure and infection by the measurement of specific host gene expression profile. The invention, therefore, will give diagnosticians the ability to diagnosis O. tsutsugamushi days or weeks earlier than previously possible with a concomitantly greater likelihood of accuracy in disease etiology. Additionally, the care provider will be able to accurately monitor the course of the disease, thereby facilitating the selection of effective drug regimens.

### SUMMARY OF INVENTION

Current methods for the detection and diagnosis of scrub typhus, caused by the rickettsial organisms Orientia tsutsugamushi early after infection are inadequate. An object of this invention is a method for diagnosis of O. tsutsugamushi early after exposure and infection to the organism and the monitoring of disease course by the modulation of expression of specific host cell genes.

A further object of the invention is the diagnosis of O. tsutsugamushi by polymerase chain reaction with low background due to amplification of contaminating DNA.

### BRIEF DESCRIPTION OF DRAWINGS

FIG. 1. Comparison of expression of interferon induced protein using mRNA from O. tsutsugamushi infected and uninfected leukocytes by real-time polymerase chain reaction.

FIG. 2. Comparison of expression of 2′-5′ oligoadenylate synthetase 3 using mRNA from O. tsutsugamushi infected and uninfected leukocytes by real-time polymerase chain reaction.

### DETAILED DESCRIPTION OF PREFERRED EMBODIMENTS

Diagnosis of the disease scrub typhus caused by O. tsutsugamushi early after exposure of individuals to the bacteria is difficult due to a lack of available assay methods. Current methods for the diagnosis of scrub typhus rely on detection of serum conversion, which is not possible until significant time has elapsed after exposure or the direct detection of the organism which requires a considerable incubation period following exposure.

Analysis of human gene expression profiles has become an increasingly important mode of predicting disease onset and for monitoring disease progression. Following exposure to external insults, such as infectious organisms or toxins, some cellular genes are modulated to increase or decrease expression. Specific cell perturbations can result in precise gene modulation profiles that are predictive for a specific external insult. The current invention capitalizes on this phenomenon by monitoring gene expression early after exposure of human cells to O. tsutsugamushi by measuring mRNA encoding the gene product or by measuring the genes protein product itself. Analysis of the gene modulation profile of cells is highly predictive of prior exposure and infection with O. tsutsugamushi. Therefore, an aspect of the invention is the detection and measurement of changes in gene expression following exposure and infection by Orientia tsutsugamushi.

Analysis of human gene expression can therefore be a predictor of infection by specific microorganisms. The general approach, therefore, of evaluating changes in human gene expression can be utilized as an effective diagnostic tool very early after infection, when other currently available methods are not effective. The approach can be used alone or in tandem with other methods, therefore, to follow progression of the disease state through treatment.

Diagnosis of infection is operationally carried out by initially measuring changes in gene expression in response to infection. Any semi-quantitatively or quantitative procedure can be used to measure changes in expression. A number of methods can be used to measure gene expression. Gene expression profiles can be measured by antibody-based methods, such as enzyme-linked immunosorbent assay (ELISA). In ELISA, a specific quantity of extracted cell protein is immobilized which is then exposed to antibody specific for genes suspected of modulation. The expression of the specific genes are normalized to the expression of a house-keeping gene. Antibody-based assays, however, suffer from the inherent requirement of antibody to selected antigens of interest and time-intensity required to conduct the assay. Therefore, alternative approaches include molecular assay methods.

Measuring changes in gene expression by reverse transcriptase polymerase (RT-PCR) chain reaction is best conducted by constructing primer sets containing at least one of the primers to the mRNA splice site. This aspect of the invention significantly increases specificity and therefore reliability of diagnosis by reducing the amplification of contaminating DNA.

Alternatively, or in addition to RT-PCR, labeled cDNA copies of mRNA from the infected human cells can be exposed to complimentary DNA copies of specific genes attached to glass microarray chips and the bound cDNA quantitated. Use of microarrays permits the convenient analysis of large numbers of genes in a single experiment. RT-PCR can also be used, in conjunction with microarray analysis, to either confirm results or to more accurately determine the relative degree of modulation of target genes. Evaluation of gene modulation profiles is conducted by computer program analysis.

Any semi-quantitatively or quantitative procedure can be used that accurately measures changes in host cell gene expression following bacterial exposure and infection. Regardless of the specific method used, the general approach in all methods employs the following steps:
a. obtaining leukocytes from blood samples from patients potentially exposed to O. tsutsugamushi;
b. extracting total RNA or protein from the leukocytes;
c. measuring gene products of a panel of important host genes by molecular, antibody-based or other methods;
d. normalizing the expression of the important host genes in the potentially infected cells to that in uninfected cells;
e. analyzing the pattern or profile of gene modulation by computer program.

Based on the gene modulation profile, a diagnosis early after exposure and infection is made by comparing the profile detected with that associated with the profile associated with O. tsutsugamushi infection. Since this method permits diagnosis much earlier after infection than other available assay methods, early, and presumably more efficacious, antibiotic treatment can be instituted. Additionally, regular re-evaluation of expressed genes during disease progression permits real-time evaluation of the effectiveness of the drug treatment regimen and modification of treatment methods, if needed. To more clearly describe the invention, the following examples are given.

### EXAMPLE 1

### Detection of Gene Expression in the in PBLs by Hybridization of Gene Products to Microarray Chips

Peripheral blood lymphocyte (PBL) were utilized as the source of RNA in order to examine the gene expression modulation in response to infection with O. tsutsugamushi. Other cell types, however could be used including purified peripheral blood mononuclear cells or subpopulations such as T-cells, B-cells and macrophages.

PBLs were obtained from whole blood from healthy individuals are drawn into cell preparation tubes containing anti-clotting agents, such as citrate. The tubes are inverted 8 to 10 times and centrifuged at 1,500×g for 30 minutes at room temperature. Plasma is then removed and the PBLs carefully removed. After washing the cells in phosphate buffered saline (PBS) the cells are suspended in RPMI 1640 media, supplemented with 2.5 mM L-glutamine, 25 mM HEPES and 7.5% human serum. The cells are then re-centrifuged and subsequently re-suspended in RPMI media until used.

The PBLs are exposed to O. tsutsugamushi by exposing the cells to the bacteria for 30 minutes in 500 μl of RPMI. Same numbers of cells are also incubated without the addition of bacteria for use in the preparation of control RNA. After 30 minutes, the cells are washed with media and re-suspended in 48 ml of complete media (RPMI supplemented with supplemented with 2.5 mM L-glutamine, 25 mM HEPES and 7.5% human serum). Five ml of the re-suspended cells was then added to flasks containing 20 ml of the RPMI media supplemented with 2.5 mM L-glutamine, 25 mM HEPES and 7.5% human serum. At specific times after infection the cultured cells are scraped off and the RNA prepared utilizing Trizol® (Invitrogen, Calsbad, Calif.). After preparation of RNA the RNA was treated with DNase to remove remaining amounts of contaminating DNA. The resulting RNA was stored at −80° C. until required.

RNA of control and O. tsutsugamushi infected cells was reverse transcribed with oligo dT to systhesize cDNAs. The cDNA was then labeled with either Cy3 or Cy5. The reference RNA (UNIVERSAL HUMAN REFERENCE RNA, Strategene, Calif.) was labeled with the dye not used in the labeling of sample cDNA. Labeled cDNA was permitted to hybridize at 42° C. overnight to glass chips containing approximately 7,680 cDNA gene sequences. The cDNA clones were spotted onto poly-L-lysine-coated slides using an OmniGrid arrayer® (GeneMachines, San Carlos, Calif.). After hybridization, the bound labeled cDNA was scanned and the image was analyzed using a GenePix® (Molecular Devices Corporation, Union City, Calif.) computer program. Normalization of induced expression, following O. tsutsugamushi infection, was conducted by comparing expression of RNA from sample PBLs to reference RNA from uninfected PBLs and subtracting from the spot intensity the background intensity to produce a channel-specific value. The data were filtered for signal intensities and background of 2.0-fold in both channels. The raw data were converted into log2 data.

For each time period, data from 2 to 4 separate experiments are obtained and the data from these multiple experiments are subjected to a 2-way ANOVA analysis. The GeneSpring® (version 5.0)(Silicon Genetics, San Carlos, Calif.) and Partek Pro® (version 5.0) (Partek, Inc, St. Charles, Mo.) are used to visualize and analyze the data, which is shown in tabular form in Table 1. Table 1 shows the mean results of two experiments using samples from three donors. A determination of up or down modultion was predicated on relative expression, in arbitrary units, either above or below a baseline level. A measurement of 1.000 would indicate no change in expression. As seen in Table 1, some genes, such as Tyrosine 3-monooxygenase, actually exhibited a large increase in expression then a precipitous decline in expression with a presumed return to baseline expression. Based on this kinetic profile, the gene was scored as up-modulated. PCR confirmatory analysis supported this contention. Gene expression that varied significantly across samples were identified as well as consistent patterns of gene expression modulation in PBLs exposed to O. tsutsugamushi. In some arrays, a scatter-plot smoother (e.g. Lowess algorithm) was employed (25).

If the PBLs had been obtained from presenting patients, early treatment, prior to that capable using currently available methods, can be initiated. Diagnosis is made by comparing and contrasting the gene modulation profile of the obtained PBLs with the expected gene induction following infection with O. tsutsugamushi.

Follow-up, confirmatory diagnostic assays, such as RT-PCR or ELISA and other antibody-based assays for the detection of bacterial antigen, can be undertaken in order to give further assurance of infection and strain identification. Furthermore, additional assays, during the course of the disease, by microarray analysis or by other traditional diagnostic methods using fresh PBLs, can be undertaken to monitor the disease progression and effectiveness of treatment.

TABLE 1

| | Relative Expression (arbitrary units) | | | | | | | |
|---|---|---|---|---|---|---|---|---|
| | Cells from uninfected | | | | Cells from infected donor | | | |
| | donor (control) | | | | (target cell) | | | |
| Gene | Time (hr) | | | | | | | |
| (up/down modulated) | 1 | 4 | 8 | 18 | 1 | 4 | 8 | 18 |
| Mylein basic Protein | 0.966 | 0.931 | 0.697 | 0.956 | 2.723 | 0.305 | 0.576 | 0.523 |
| (down) | | | | | | | | |
| Lymphotoxin alpha (up) | — | — | — | — | 1.33 | 1.148 | 1.230 | 5.807 |
| FK506 binding protein (up) | — | — | — | — | 1.140 | 4.654 | 2.145 | 1.656 |
| Interferon induced protein | — | — | — | — | 0.830 | 2.234 | 9.999 | 2.353 |
| with tetracopeptide repeats | | | | | | | | |
| 2 (IFIT2) (up) | | | | | | | | |
| Chemokine receptor 7 (up) | 0.894 | 0.813 | 1.302 | 0.821 | 2.614 | 3.367 | 0.958 | 1.025 |
| Never-in-Mitosis gene a | — | — | — | — | 12.415 | 2.014 | 0.776 | 3.230 |
| relaed kinase (NIMA) (up) | | | | | | | | |
| Chemokine ligand 3 | — | — | — | — | 3.100 | 1.723 | 10.812 | 1.863 |
| (CCL3) (up) | | | | | | | | |
| Transcription factor 12 | — | — | — | — | 0.0 | 1.044 | 4.133 | 7.138 |
| (up) | | | | | | | | |
| Minichromosome maint | — | — | — | — | 1.534 | 0.785 | 5.204 | 4.484 |
| deficient 3 associted | | | | | | | | |
| protein (up) | | | | | | | | |
| NADH dehydrogenase Fe—S | — | — | — | — | 1.352 | 2.191 | 1.494 | 3.909 |
| Protein 3 | | | | | | | | |
| Zinc finger protein 147 | — | — | — | — | 2.091 | 2.028 | 1.440 | 7.780 |
| (ZNF 147) (up) | | | | | | | | |
| Chemokine ligand 8 | — | — | — | — | 0.616 | 1.069 | 16.370 | 6.687 |
| (CCL8) (up) | | | | | | | | |
| 2′-5′ oligoadenylate synt 3 | — | — | — | — | 0983 | 3.945 | 8.410 | 3.401 |
| (up | | | | | | | | |
| TP inducible gene | 0.939 | 0.876 | 1.015 | 0.982 | 0.588 | 2.208 | 1.577 | 0.510 |
| (TP53TG3) (down) | | | | | | | | |
| Junction plakoglobin (JUP) | — | — | — | — | 22.99391 | 1.445 | 2.654 | 1.909 |
| (up) | | | | | | | | |
| Viperin (cig5) (up) | — | — | — | — | 2.868 | 0.666 | 8.112 | 4.033 |
| Replication Protein A2 (up) | 0.971 | 0.914 | 0.954 | 0.955 | 1.721 | 0.969 | 0.938 | 1.043 |
| G protein signaling 1 | 0.800 | 0.896 | 0.404 | 0.935 | 0.218 | 1.006 | 5.104 | 1.104 |
| (RGS1) (up) | | | | | | | | |
| Apoptosis-related cysteine | 0.956 | 0.546 | 1.141 | 0.932 | 0.858 | 1.491 | 1.055 | 1.023 |
| protease (CASP7) (up) | | | | | | | | |
| Tyrosine 3- | 0.973 | 0.918 | 0.786 | 0.758 | 3.447 | 0.472 | 0.659 | 0.660 |
| monooxygenase/tryptophan | | | | | | | | |
| 5-monooxygenase | | | | | | | | |
| activation protein, beta | | | | | | | | |
| polypeptide (up) | | | | | | | | |
| Polymerase gamma 2, | 0.746 | 0.936 | 0.840 | 0.721 | 1.631 | 0.738 | 0.657 | 1.069 |
| accessory subunit (up) | | | | | | | | |
| Enhancer of zeste homolog | 0.934 | 0.903 | 1.068 | 0.865 | 1.699 | 0.942 | 0.906 | 1.091 |
| 2 (EZH2) (up) | | | | | | | | |

### EXAMPLE 2

### Analysis of Gene Modulation by Polymerase Chain Reaction

Gene modulation can be determined by quantitative reverse transcriptase polymerase chain reaction (RT-PCR). RT-PCR analysis can also be used alone or in tandem with other methods, such as microarray analysis, in order to confirm the results obtained by that method. In this embodiment, human monocytic cells or PBLs are obtained from potentially O. tsutsugamushi infected patients. Whole blood from healthy individuals are drawn into cell preparation tubes containing anti-clotting agents, such as citrate. The tubes are inverted 8 to 10 times and centrifuged at 1,500×g for 30 minutes at room temperature. Plasma is then removed and the PBLs carefully removed. After washing the cells in phosphate buffered saline (PBS) the cells are suspended in RPMI 1640 media, supplemented with 2.5 mM L-glutamine, 25 mM HEPES and 7.5% fetal calf serum. The cells are then re-centrifuged and subsequently re-suspended in RPMI media until used.

A specific example of how the PCR is practiced in the diagnosis of O. tsutsugamushi is illustrated by the following example. Freshly isolated human leukocytes are either used uninfected or infected with O. tsutsugamushi at MOI of 1.0 for 45 minutes at 35° C. The leukocytes, both infected and uninfected, were culture at 35° C. in 5% CO₂. After incubation, infected and uninfected leukocytes were collected at 1 hour, 4 hours, 8 hours or 18 hours after infection. Total RNA was then obtained according to the Trizo® method (Invitrogen, Carlsbad, Calif.) followed by treatment with DNase. The concentration of total RNA in each sample was initially quantitated spectrophotometrically at 260 nm. The first strand cDNA synthesis was performed in 100 μl reaction volumes containing 15 μg of total RNA from each sample and 2 μl of oligo dT or random primers is added to total RNA and the mixture heated at 70° C. for 5 minutes then cooled on ice for 3 minutes. Following the addition of the first strand buffer, dNTP, DTT and reverse transcriptase, the final mixture was incubated at 42° C. for 50 minutes and then shifted to 70° C. for 10 minutes. Specific primers that flank the mRNA splicing sites of 658 genes were designed using a primer designing algorithm, GeneLooper™, (GeneHarbor, Inc, Rockville, Md.). The primers were designed to provide a uniform annealing temperature of 62° C. and amplification products of 300-350 bp. Synthesized cDNA was semi-quantitated by PCR in “comparing groups” composed of cDNA derived uninfected and infected leukocytes. Semi-quantitation was normalized to the house-keeping gene glyceraldehyde 3-phosphate dehydrogenase. Synthesized cDNA was stored at −80° C. until needed.

After production of a cDNA copy of the RNA by reverse transcriptase, primers to selected targets are used to amplify specific target genes sequences. Primer sets are designed such that at least one primer member of a primer set is complementary to the sequence encoding the splice site of the target mRNA. Targeting of primer sequences complementary to splice junctions ensures that amplification of sequences will not occur using genomic DNA as template. Thus background amplification due to amplification of remaining DNA, despite treatment of RNA with DNase will be minimized.

PCR analysis was performed using the i-cycler™ (BioRad, Hercules, Calif.) using cDNA from infected and uninfected leukocytes. PCR was performed using the light cycler DNA master SYBR green I® kit (Rocke Diagnositcis, Indianapolis, Ind.) in 20 μl reaction volumes using cDNA from infected and uninfected comparing groups, after addition of dNTPs, PCR buffer, forward and reverse primers and Taq polymerase. The PCR mixture was incubated at 94° C. for 3 min followed by 32 cycles of 3 step amplification at 94° C. for 30 seconds, 62° C. for 30 seconds and 72° C. for 1 minute. A 72° C. hold step was performed for 5 minutes following PCR cycling. PCR amplified products of reverse transcribed RNA can be visualized by first separating the products by electrophoresis on 1% agarose gel. Semi-quantitation of the PCR gel image data was then performed using gel analyzing software (GelPicAnalyzer™, GeneHarbor, Inc., Rockville, Md.). Background was subtracted and the values normalized to the amplified house-keeping gene (i.e. glyceraldehyde 3-phosphate dehydrogenase). The results of these studies are illustrated in Table 2.

The open reading frame (ORF) of two genes, interferon induced protein and 2′-5′ oligoadenylate synthetase 3, were used to develop a fluorogenic-based PCR assay. For the assay PCR probes were labeled with 6-carboxyfluorescein (FAM) and 6-carboxytetramethyl-rhodamine (TAMRA) at the 5′ end and 3′ ends, respectively. This permits monitoring of specific PCR product over time.

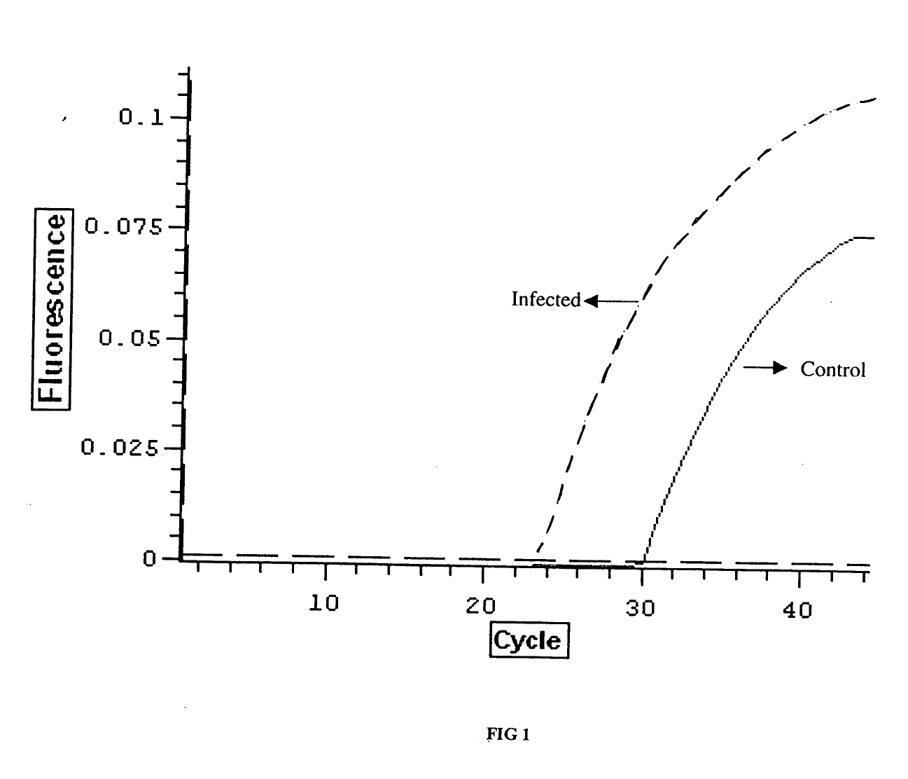

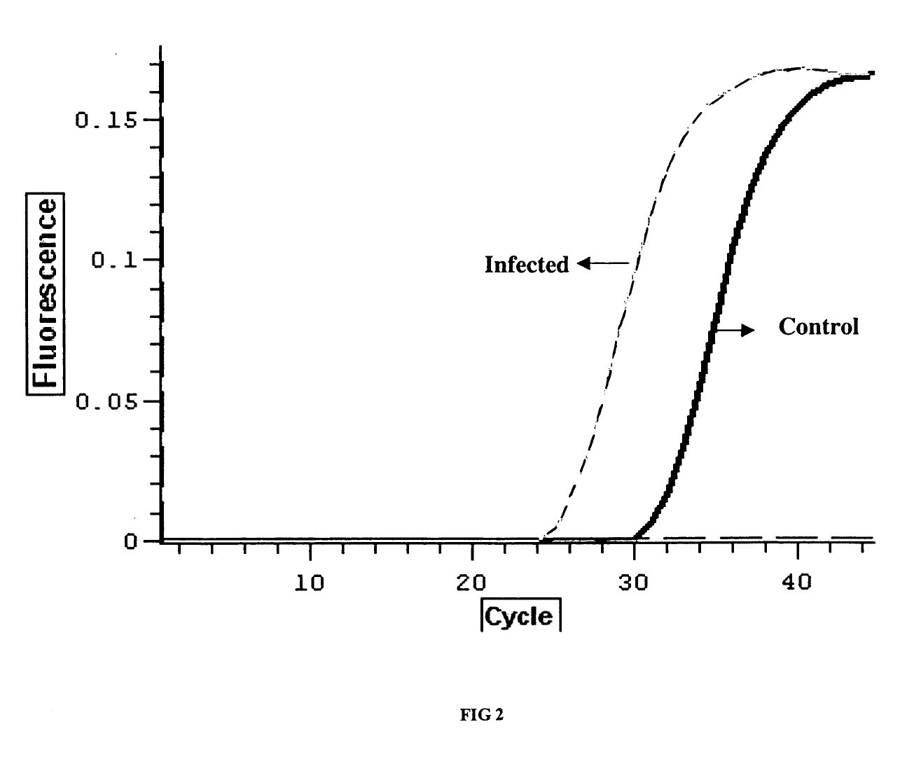

PCR reactions were conducted in 25-50 μl reaction mixtures containing 1×TaqMan Universal PCR Master Mix®), 0.02 μM of each primer, 0.1 μM probe and 1 μl of diluted cDNA template. After incubation at 50° C. for 2 min and denaturation at 95° C. for 10 minutes the reaction is permitted to proceed for 45 to 60 cycles with denaturation at 94° C. for 15 seconds and extension at 60° C. for 1 minute. The 18 S rRNA gene was included as an endogenous reference and the comparative Cₜ (threshold cycles) method applied using arithmetic formulas. By this method, the amount of target was normalized to that of the 18S RNA. Real-time RT-PCR assays are performed in triplicate for each sample and a mean value and standard deviation calculated for relative RNA expression levels. The results of studies using interferon induced protein and 2′-5′ oligoadenylate synthetase 3 as target genes is shown in FIG. 1 and FIG. 2, respectively. As can be seen in FIG. 1 and FIG. 2, a marked shift in the number of cycles required for visualization of product is clearly evident in the mRNA from infected (i.e. approximately 14 to 15 cycles) verses uninfected cells (i.e. 30 to 31 cycles) in these two genes. In both situations, the 18 S RNA was used to normalize the raw PCR reaction data.

Analysis of only interferon induced protein and 2′-5′ oligoadenylate synthetase 3 from patients suspected of infection, compared mRNA from uninfected leukocytes, is required in order to make a determination of prior infection. However, analysis of these genes in combination with other genes that were evaluated in Tables 1 and 2 would provide a more accurate analysis of and determination of prior infection.

TABLE 2

| | Sample | Up/down | Donor 1 | | Donor 2 | | Donor 3 | |
|---|---|---|---|---|---|---|---|---|
| Gene | number | regulated | Control | Infected | Control | Infected | Control | Infected |
| Mylein Protein zero | 1 | down | 1,124 | 338 | 8,434 | 5,441 | 4,384 | 3,176 |
| | 2 | down | 10,663 | 3,781 | 7,744 | 7,338 | 3,725 | 2,872 |
| Lymphotoxin alpha | 1 | up | — | — | 6,812 | 11,698 | 11,077 | 28,815 |
| | 2 | up | 0 | 8,618 | 1,085 | 12,586 | — | — |
| FK506 binding protein | 1 | up | — | — | 3,065 | 10,803 | 15,877 | 21,439 |
| | 2 | up | 3,065 | 11,137 | 11,796 | 14,793 | — | — |
| Interferon induced protein | 1 | up | — | — | 5,114 | 15,063 | 15,707 | 27,989 |
| with tetratricopeptide | | | | | | | | |
| repeats 2 (IFIT2) | | | | | | | | |
| | 2 | up | 4,228 | 16,620 | 0 | 963 | — | — |
| Chemokine receptor 7 | 1 | up | 3,358 | 9,751 | 20,227 | 37,167 | 11,462 | 14,687 |
| (CCR7) | | | | | | | | |
| | 2 | up | 6,430 | 16,156 | 15,070 | 20,887 | 8,731 | 17,533 |
| Never in mitosis gene a- | 1 | up | — | — | 502 | 8,625 | 13,677 | 22,057 |
| related kinase 3 | | | | | | | | |
| | 2 | up | — | — | 13,626 | 20,958 | — | — |
| Chemokine ligand 3 | 1 | up | — | — | 58 | 883 | 15,266 | 22,297 |
| (CCL3) | | | | | | | | |
| | 2 | up | — | — | 9,548 | 11,990 | — | — |
| Transcription factor 12 | 1 | up | — | — | 9,280 | 13,089 | 874 | 16,857 |
| | 2 | up | 9,280 | 13,442 | 9,390 | 14,878 | — | — |
| Minichromosome maint | 1 | up | — | — | 3,622 | 4,104 | 5,898 | 18,194 |
| deficient 3 associated | | | | | | | | |
| protein | | | | | | | | |
| | 2 | up | 2,814 | 11,120 | 7,971 | 9,619 | — | — |
| NADH dehydrogenase Fe—S | 1 | up | — | — | 10,323 | 17,684 | 16,501 | 24,291 |
| Protein 3 | | | | | | | | |
| | 2 | up | 10,323 | 18,230 | 9,482 | 14,189 | — | — |
| Zinc finger protein 147 | 1 | up | — | — | 16,446 | 20,901 | 10,352 | 25,411 |
| (ZNF 147) | | | | | | | | |
| | 2 | up | 0 | 16,180 | 2,872 | 13,498 | — | — |
| Chemokine ligand 8 | 1 | up | — | — | 12,444 | 19,140 | 4,229 | 15,296 |
| (CCL8) | | | | | | | | |
| | 2 | up | 4,092 | 5,282 | 433 | 15,670 | — | — |
| 2′-5′ oligoadenylate synt 3 | 1 | up | — | — | 2,596 | 14,787 | 3,084 | 18,794 |
| | 2 | up | 4,092 | 18,919 | 0 | 9,299 | — | — |
| TP inducible gene | 1 | down | 1,838 | 1,138 | 8,458 | 3,481 | 2,761 | 2,490 |
| (TP53TG3) | | | | | | | | |
| | 2 | down | 6,970 | 2,757 | 5,104 | 6,629 | 21,700 | 14,857 |
| | Sample | Up/down | Donor 1 | | Donor 2 | | Donor 3 | |
| Gene | number | regulated | Control | Treatment | Control | Treatment | Control | Treatment |
| Junction plakoglobin (JUP) | 1 | up | 1 | 1 | 6,355 | 4,833 | 1 | 3,179 |
| | 2 | up | 1 | 7,559 | 0 | 7,731 | 1 | 1 |
| Viperin (cig5) | 1 | up | — | — | 16,014 | 19,723 | 20,773 | 27,480 |
| | 2 | up | 8,512 | 15,357 | 16,670 | 24,589 | — | — |
| Replication Protein A2 | 1 | up | 6,054 | 34,550 | 22,858 | 23,905 | 17,386 | 23,297 |
| (RPA2) | | | | | | | | |
| | 2 | up | 3,015 | 4,644 | 8,226 | 6,428 | — | — |
| G protein signaling 1 | 1 | up | 17,053 | 26,272 | 18,312 | 21,610 | 11,945 | 11,805 |
| (RGS1) | | | | | | | | |
| | 2 | up | 18,486 | 26,449 | — | — | — | — |
| Apoptosis-related cysteine | 1 | up | 4,471 | 15,834 | 3,086 | 4,124 | 0.0 | 10,148 |
| protease (CASP7) | | | | | | | | |
| | 2 | up | 13,281 | 14,530 | — | — | — | — |
| Tyrosine 3- | 1 | up | 29,277 | 35,740 | 23,164 | 25,013 | 18,072 | 26,616 |
| monooxygenase/tryptophan | | | | | | | | |
| 5-monooxygenase | | | | | | | | |
| activation protein, beta | | | | | | | | |
| polypeptide | | | | | | | | |
| | 2 | up | 5,228 | 14,320 | 1,524 | 2,567 | — | — |
| Polymerase gamma 2, | 1 | up | 0.0 | 11,309 | 16,683 | 19,821 | 15,972 | 21,288 |
| accessory subunit | | | | | | | | |
| | 2 | up | 14,467 | 20,818 | 1,148 | 8,158 | — | — |
| Enhancer of zeste homolog | 1 | up | 13,362 | 14,065 | 17,005 | 22,224 | 18,024 | 22,360 |
| 2 (EZH2) | | | | | | | | |
| | 2 | up | 5,470 | 19,153 | 5,953 | 9,207 | — | — |

### REFERENCES

1. Brown, G. W., D. M. Robinson, D. L. Huxsoll, T. S. Ng, K. J. Lim and G. Sannasey. 1976. Scrub typhus: a common cause of illness in indigenous populations. Trans. R. Soc. Trop. Med. Hyg. 70:444-448.
2. Brown, G. W., J. P. Saunders, S. Singh, D. L. Huxsoll, and A. Shirai. 1978. Single dose doxycycline therapy for scrub typhus. Trans. R. Soc. Trop. Med. Hyg. 72:412-416.
3. Bozemean, F. M., And B. L. Elisberg. 1963. Serological diagnosis of scrub typhus by indirect immunofluorescence. Proc. Soc. Exp. Biol. Med. 112:568-573.
4. Dasch, G. A., S. Halle, and A. L. Bourgeois. 1979. Sensitive microplate enzyme-linked immunosorbent assay for detection of antibodies against the scrub typhus rickettsia, Rickettsia tsutsugamushi. J. clin. Microbiol. 9:38-48.
5. Dohany, A. L., A. Shirai, D. M. Robinson, S. Ram, and D. L. Huxsoll. 1978. Identification and antigenic typing of Rickettsia tsutsugamushi in naturally infected chiggers (Acarina: Trombiculidae) by direct immunofluorescence. Am. J. Trop. Med. Hyg. 27:1261-1264.
6. Kelly, D. J., P. W. Wong, E. Gan and G. E. Lewis, Jr. 1988. Comparative evaluation of the indirect immunoperoxidase test for the serodiagnosis of rickettsial disease. Am. J. Trop. Med. Hyg. 38:400-406.
7. Suto, T. 1980. Rapid serological diagnosis of tsutsugamushi disease employing the immuno-peroxidase reaction with cell cultured ricketsia. Clin. Viol. 8:425-429.
8. Suwanabun, N., C. Chouriyagune, C. Eamisila, P. Watcharapichat, G. A. Dasch, R. S. Howard and D. J. Kelly. 1997. Evaluation of an enzyme-linked immunosorbet assay in Thai scrub typhus patients. Am. J. Trop. Med. Hyg. 56:38-43.
9. Weddle, J. R., T. C. Chan, K. Thompson, H. Paxton, D. J. Kelly, G. Dasch, and D. Strickman. 1995. Effectiveness of a dot-blot immunoassay of anti-Rickettsia tsutsugamushi antibodies for serologic analysis of scrub typhus. Am. J. Trop. Med. Hyg. 53:43-46.
10. Yamamoto, S., and Y. Minamishima. 1982. Serodiagnosis of tsutsugamushi fever (scrub typhus) by the indirect immunoperoxidase technique. J. Clin. Microbiol. 15:1128-1132.
11. Murata, M. Y. Yoshida, Osono, N. Ohashi, Oyanagi, H. Urakami, A. Tamura, S. Nogami, H. Tanaka, and A. Kawamura, Jr. 1986. Production and characterization of monoclonal strain-specific antibodies against prototype strains of Rickettsia tsutsugamushi. Microbiol. Immunol. 30:599-610.
12. Furuya, Y., Y. Yoshida, T. Katayama, F. Kawamori, S. Yamamoto, N. Ohashi, A. Kamura and A. Kawamura, Jr. 1991. Specific amplification of Rickettsia tsutsugamushi DNA from clinical specimen by polymerase chain reaction. J. Clin. Microbiol. 29:2628-2630.
13. Kelly, D. J., G. A. Dasch, T. C. Chye, and T. M. Ho. 1994. Detection and characterization of Rickettsia tsutsugamushi (Rickettsiales: Rickettsiaceae) in infected Leptotrombidium (Leptotrombidium) fletcheri chiggers (Acari: Trombiculidae) with the polymerase chain reaction. J. Med. Entomol. 31:691-699.
14. Dasch, G. A., D. Strickman, G. Watt, and C. Eamsila. 1996. Measuring genetic variability in Orientia tsutsugamushi by PCR/RFLP analysis: a new approach to questions about its epidemiology, evolution and ecology, p. 79-84. In J. Kazar (ed.) Rickettsiae and Rickettsial Diseases. Vth International Symposium. Slovak Academy of Sciences, Bratislava.
15. Enatsu, T., H. Urakami, A. Tamura. 1999. Phylogenetic analysis of Orientia tsutsugamushi strains based on the sequence homologies of 56-kDa type-specific antigen genes. FEMS 180:163-169.
16. Horinouchi, H., K. Murai, A. Okayama, Y. Nagatomo, N. Tachibana, and H. Tsubouchi. 1996. Genotypic identification of Rickettsia tsutsugamushi by restriction fragment length polymorphism analysis of DNA amplified by polymerase chain reaction. Am. J. Trop. Med. Hyg. 54:647-651.
17. Ohashi, N., Y. Koyama, H. Urakami, M. Fukuhara, A. Tamura, F. Kawamori, S. Yamamoto, S. Kasuya, and K. Yoshimura. 1996. Demonstration of antigenic and genotypic variation in Orientia tsutsugamushi which were isolated in Japan, and their classification into type and subtype. Microbiol. Immunol. 40:627-638.
18. Tamura, A., N. Ohashi, Y. Koyama, M. Fukuhara, F. Kawamori, M. Otsuru, P-F. Wu, and S-Y. Lin. 1997. Characterization of Orientia tsutsugamushi isolated in Taiwan by immunofluoescence and restriction fragment length polymorphism analyses. FEMS Microbiol. Lett. 150:225-231.
19. Jett, M. Alterations in Gene Expression Show Unique Patterns in Response to Toxic Agents. 21st Army Science Conference, Proceedings 21:529-534.
20. Nau, G. J., J. F. L. Richmon, A. Schlesinger, E. G. Jennings, E. S. Lander and R. A. Young. 2002. Human macrophage activation programs induced by bacterial pathogens. Proc. Natl. Acad. Sci. (USA) vol 99 (3):1503-1508.
21. Lockhart D J and E. A. Winzeler. 2000. Genomics, gene expression and DNA arrays. Nature. 405:827-836.
22. Eckmann L, J. R. Smith, M. P. Housley, M. B. Dwinell and M. F. Kagnoff. 2000. Analysis by high density cDNA arrays of altered gene expression in human intestinal epithelial cells in response to infection with the invasive enteric bacteria Salmonella. J. Biol. Chem. 275(19): 14084-14094.
23. Blader I J, I. D. Manger, and J. C. Boothroyd. 2001. Microarray analysis reveals previously unknown changes in Toxoplasma gondii-infected human cells. J. Biol. Chem. 276:24223-24231.
24. Ge, H., Y.-Y. Chuang, E., S. Zhao, J. J. Temenak, W.-M. Ching, W.-M. 2004. Comparative genomics of Rickettsia prowazekii Madrid E and Breinl strains. J. Bacteriol 186 (2):556-565.
25. Yang, Y. H., Dudoit, S., Luu, P., Lin, D. M., Peng, V., Ngai, J. and Speed, T. P. 2002. Normalization for cDNA microarray data: A robust composite method addressing single and multiple slide systematic variation. Nucleic Acids Research 30:e15.

Having described the invention, one of skill in the art will appreciate in the claims that many modifications and variations of the present invention are possible in light of the above teachings. It is therefore, to be understood that, within the scope of the claims, the invention may be practiced otherwise than as specifically described.

What is claimed is:

1. A method for the early diagnosis of Orientia tsutsugamushi infection wherein diagnosis is by determining a gene expression profile comprising the steps:
a. obtaining total RNA from cells from a patient, total RNA from uninfected control cells and RNA from cells infected with Orientia tsutsugamushi;
b. measuring the expression of genes from said patient cells, uninfected control cells and Orientia tsutsugamushi infected cells to obtain gene expression profile comprising the genes; lymphotoxin alpha, FK506 binding protein, interferon induced protein with tetratricopeptide repeats 2, chemokine receptor 7, never-in-mitosis gene a-related kinase 3, chemokine ligand 3, transcription factor 12, minichromosome maintenance deficient 3 associated protein, NADH dehydrogenase Fe—S protein 3, zinc finger protein 147, chemokine ligand 8,2′-5′ oligoadenylate synthetase 3, junction plakoglobin, viperin, replication protein A2, G protein signaling 1, apoptosis-related cysteine protease, tyrosine 3-monooxygenase/tryptophan 5-monooxygenase activation protein, beta polypeptide, polymerase gamma 2 accessory subunit, enhancer of zeste homology 2 and to the expected gene profile of genes expected to be repressed including: myelin protein zero, TP inducible gene;
c. determining the modulation of said expression of said genes by comparing the expression of said patient genes with the expression of said genes from said infected and uninfected cells;
d. creating a profile of the modulation of said patient, infected and uninfected cell genes;
e. comparing said patient cell gene modulation profile to the profile to the profile of said infected and uninfected cell genes.

2. The method of claim 1, wherein said infected, uninfected and patient cells are selected from the group consisting of leukocytes, peripheral blood lymphocytes and mononuclear cells.

3. The method of claim 2, wherein said measurement of gene expression is by microarray analysis comprising the steps:
a. synthesizing a cDNA copy of said RNA with a labeled;
b. hybridizing said labeled cDNA to DNA sequences immobilized on microarray chips encoding said genes expected to be induced and repressed following Orientia tsutsugamushi infection;
c. measuring the amount of hybridization of said labeled cDNA to obtain said patient gene profile;
d. comparing said patient gene profile to said expected gene profile.

4. The method of claim 3 wherein said label is a fluor selected from the group consisting essentially of Cy3 and Cy5.

5. The method of claim 2, wherein said measuring of expression is by reverse transcriptase polymerase chain reaction.

6. The method of claim 5 wherein the primers sets for said reverse transcriptase polymerase chain reaction contain a primer complementary to the sequence encoding the splice site of the target mRNA.

7. The method of claim 6, wherein said reverse transcriptase polymerase chain reaction comprising the steps:
a. synthesizing a cDNA copy of said RNA;
b. amplifying said cDNA by polymerase chain reaction using forward and reverse primers to a control house-keeping gene and to one or more genes including: lymphotoxin alpha, FK506 binding protein, interferon induced protein with tetratricopeptide repeats 2, chemokine receptor 7, never-in-mitosis gene a-related kinase 3, chemokine ligand 3, transcription factor 12, minichromosome maintenance deficient 3 associated protein, NADH dehydrogenase Fe—S protein 3, zinc finger protein 147, chemokine ligand 8,2′-5′ oligoadenylate synthetase 3, junction plakoglobin, viperin, replication protein A2, G protein signaling 1, apoptosis-related cysteine protease, tyrosine 3-monooxygenase/tryptophan 5-monooxygenase activation protein, beta polypeptide, polymerase gamma 2 accessory subunit, enhancer of zeste homology 2, myelin protein zero and TP inducible;
c. separating polymerase chain reaction products by gel electrophoresis;
d. measuring the relative expression of said electrophoresis separated products.

8. The method of claim 6, wherein said reverse transcriptase polymerase chain reaction is real-time reverse transcriptase polymerase chain reaction comprising the steps:
a. synthesizing a reporter dye and quencher dye labeled cDNA copy of said RNA;
b. amplifying said cDNA using forward and reverse primers specific to a control gene and one or more genes including: lymphotoxin alpha, FK506 binding protein, interferon induced protein with tetratripeptide repeats 2, chemokine receptor 7, never-in-mitosis gene a-related kinase 3, chemokine ligand 3, transcription factor 12, minichromosome maintenance deficient 3 associated protein, NADH dehydrogenase Fe—S protein 3, zinc finger protein 147, chemokine ligand 8,2′-5′ oligoadenylate synthetase 3, junction plakoglobin, viperin, replication protein A2, G protein signaling 1, apoptosis-related cycteine protease, tyrosine 3-monooxygenase/tryptophan 5-monooxygenase activation protein, beta polypeptide, polymerase gamma 2 accessory subunit, enhancer of zeste homology 2 and to the expected gene profile of genes expected to be repressed including: myelin protein zero, TP inducible gene;
c. determining the number of polymerase chain reaction cycles required for detection of said reporter dye;
d. comparing said number of polymerase chain reaction cycles required for detection between said patient RNA, RNA from infected and RNA from uninfected cells.

9. The method of claim 8, wherein said reporter dye is 5′-FAM and said quencher dye is 3′-TAMRA.

10. The method of claim 2, wherein said measurement of gene expression is by enzyme-linked immunosorbent assay comprising the steps:
a. extracting total protein from said cells;
b. immobilizing specific quantities of said total protein and exposing each of said immobilized quantity of total protein to an antibody specific for a house keeping gene and one or more of the genes including: lymphotoxin alpha, FK506 binding protein, interferon induced protein with tetratricopeptide repeats 2, chemokine receptor 7, never-in-mitosis gene a-related kinase 3, chemokine ligand 3, transcription factor 12, minichromosome maintenance deficient 3 associated protein, NADH dehydrogenase Fe—S protein 3, zinc finger protein 147, chemokine ligand 8,2′-5′ oligoadenylate synthetase 3, junction plakoglobin, viperin, replication protein A2, G protein signaling 1, apoptosis-related cysteine protease, tyrosine 3-monooxygenase/tryptophan 5-monooxygenase activation protein, beta polypeptide, polymerase gamma 2 accessory subunit, enhancer of zeste homology 2, myelin protein zero and TP inducible;
c. measuring the relative expression of said genes by measuring the binding of specific antibody to said gene product and said house-keeping gene product.
